(19) United States
(12) Patent Application Publication
(10) Pub. No.: US 2020/0163682 A1
(43) Pub. Date: May 28, 2020

(54) DISC PREPARATION TOOL

(71) Applicant: CTL Medical Corporation, Addison, TX (US)

(72) Inventors: Jon SUH, Ambler, PA (US); Sean SUH, Milltown, NJ (US)

(21) Appl. No.: 16/696,680

(22) Filed: Nov. 26, 2019

Related U.S. Application Data
(60) Provisional application No. 62/771,371, filed on Nov. 26, 2018.
(60) Provisional application No. 62/772,881, filed on Nov. 29, 2018.

(51) Int. Cl. A61B 17/16

(52) U.S. Cl. CPC A61B 17/1608; A61B 2017/00526; A61B 17/1671

(57) ABSTRACT

Disclosed are devices and methods for use in the surgical treatment of vertebrae and/or other bones, particularly tools for cutting and/or removing soft and/or hard tissues.

### CROSS-REFERENCE TO RELATED APPLICATIONS

This application claims priority to and benefit of U.S. Provisional Patent Application No. 62/771,371 entitled “DISC PREPARATION TOOL (PITUITARY TYPE),” filed Nov. 26, 2018, and U.S. Provisional Patent Application No. 62/772,881 entitled “DISC PREPARATION TOOL (PITUITARY TYPE),” filed Nov. 29, 2018. The disclosures of each of these references are incorporated by reference herein in their entireties.

### FIELD OF THE INVENTION

The present subject matter relates generally to devices and methods for removing soft tissues and/or preparing adjacent vertebrae or other bones for orthopedic implants including, but not limited to, spinal implants for disc replacement surgeries and/or arthroplasty procedures.

### BACKGROUND OF THE INVENTION

The spinal column of vertebrates provides support to bear weight and protection to the delicate spinal cord and spinal nerves. The spinal column includes a series of vertebrae stacked on top of each other. There are typically seven cervical (neck), twelve thoracic (chest), and five lumbar (low back) segments. Each vertebra has a cylindrical shaped vertebral body in the anterior portion of the spine with an arch of bone to the posterior, which covers the neural structures. Between each vertebral body is an intervertebral disk, a cartilaginous cushion to help absorb impact and dampen compressive forces on the spine. To the posterior, the laminar arch covers the neural structures of the spinal cord and nerves for protection. At the junction of the arch and anterior vertebral body are articulations to allow movement of the spine.

Various types of problems can affect the structure and function of the spinal column. These can be based on degenerative conditions of the intervertebral disk or the articulating joints, traumatic disruption of the disk, bone or ligaments supporting the spine, tumor or infection. In addition, congenital or acquired deformities can cause abnormal angulation or slippage of the spine. Anterior slippage (spondylolisthesis) of one vertebral body on another can cause compression of the spinal cord or nerves. Patients who suffer from one of more of these conditions often experience extreme and debilitating pain and can sustain permanent neurological damage if the conditions are not treated appropriately.

Various physical conditions can manifest themselves in the form of damage or degeneration of an intervertebral disc, the result of which is mild to severe chronic back pain. Intervertebral discs serve as “shock” absorbers for the spinal column, absorbing pressure delivered to the spinal column. Additionally, they maintain the proper anatomical separation between two adjacent vertebrae. This separation is necessary for allowing both the afferent and efferent nerves to exit and enter, respectively, the spinal column. Alternatively, or in addition, there are several types of spinal curvature disorders. Examples of such spinal curvature disorders include, but need not be limited to, lordosis, kyphosis and scoliosis.

One technique of treating spinal disorders, in particular the degenerative, traumatic and/or congenital issues, is via surgical arthrodesis of the spine. This can be accomplished by removing the intervertebral disk and replacing it with implant(s) and/or bone and/or immobilizing the spine to allow the eventual fusion or growth of the bone across the disk space to connect the adjoining vertebral bodies together. The stabilization of the vertebra to allow fusion is often assisted by the surgically implanted device(s) to hold the vertebral bodies in proper alignment and allow the bone to heal, much like placing a cast on a fractured bone. Such techniques have been effectively used to treat the above-described conditions and in most cases are effective at reducing the patient's pain and preventing neurological loss of function.

Treatment for a diseased or damaged disc can involve partial or complete removal of the affected disc and implantation of a spinal disc replacement or spinal spacer for fusion or non-fusion purposes. A discectomy to remove a spinal disc can involve multiple passes into an out of the disc space between vertebrae, and each one of these passes to remove the disc tissue can increase the risk of nerve injury and contamination of the disc space. In addition, each pass becomes time consuming for the surgeon as there must be a focus with each pass on proper entry into the disc space, removing a portion of tissue, and manually clearing the device of the removed tissue to prepare the device for the subsequent pass into the disc space. During these procedures, it can also be advantageous to prepare the vertebral endplates for the implantation of a disc implant, which is routinely achieved using a separate tool. In addition, spinal curvature disorders and/or contour issues present on the surfaces of the vertebrae may present additional challenges. As such, there is need for further improvement, and the present subject matter is such improvement.

### BRIEF SUMMARY OF THE INVENTION

The following presents a simplified summary of the subject matter in order to provide a basic understanding of some aspects of the subject matter. This summary is not an extensive overview of the subject matter. It is intended to neither identify key or critical elements of the subject matter nor delineate the scope of the subject matter. Its sole purpose is to present some concepts of the subject matter in a simplified form as a prelude to the more detailed description that is presented later.

In accordance with an aspect of the present subject matter, devices and methods for removing soft and/or hard tissue material such as an intervertebral disc and/or preparing an intervertebral disc space are provided. In one embodiment, a spinal disc preparation tool is provided and includes a pair of elongate shafts, each shaft having one of an upper jaw element and a lower jaw element positioned on a distal end thereof. The upper and lower jaw elements are pivotally movable relative to each other. The upper and lower jaws include one or more cutting surfaces or edges disposed therein.

In other embodiments, the spinal disc preparation tool can include a handle coupled to a proximal end of the elongate shaft (s), having an actuator operatively associated with the upper and/or lower jaws for pivoting or otherwise angling one or both of the jaws relative to the tool, or relative to each other. The upper and/or lower jaws of the disclosed devices can include a plurality of cutting surfaces and/or one or more abutment surfaces.

In another embodiment, a spinal disc preparation tool is provided and includes one or more elongate shaft(s), with an upper jaw movably coupled to the elongated shaft, and a lower jaw. At least one tissue cutting surface feature is formed on at least one of the upper and lower jaws. The tissue cutting surface feature can be configured in a variety of ways, such as to remove tissue along a surface of a vertebral endplate or to cut into a surface of an endplate. The surface feature can also be formed in a number of ways. For example, the at least one tissue cutting surface feature can be in the form of a rasp formed on a surface of the lower jaw, a curette formed on a surface of the lower jaw, a tooth formed on one or more surfaces of the upper and/or lower jaws, or a protrusion formed on an exterior surface of the lower jaw and having a concavity for scraping tissue.

In another embodiment, a spinal disc preparation tool is provided and includes at least one elongate shaft and upper and lower jaws disposed on a distal end of the elongate shaft. The upper jaw is movable relative to the lower jaw, and includes a surface to cut tissue. At least one of the upper and lower jaws and/or the cutting surfaces therein are removable and replaceable. In one embodiment, one or both of the jaws can include a removable hood or a removable insert having a blade thereon for cutting tissue. In another exemplary embodiment, the upper and lower jaws can be coupled to a distal portion of the elongate shaft, and the distal portion of the elongate shaft can be removably coupled to a proximal portion of the elongate shaft such that the distal portion of the elongate shaft and the upper and lower jaws are removable and replaceable.

Various surgical methods for cutting tissue are also provided, and in one embodiment the method can include inserting a distal end of an elongate shaft of a spinal disc preparation tool between adjacent vertebrae, and actuating one or more of the jaws on the distal end of the elongate shaft to move the upper jaw relative to a lower jaw on the distal end of the elongate shaft to cut tissue on the adjacent vertebrae.

In accordance with another aspect of the present subject matter, a method for manufacturing devices as set for within any of the details described with the present application is provided.

While embodiments and applications of the present subject matter have been shown and described, it would be apparent that other embodiments, applications and aspects are possible and are thus contemplated and are within the scope of this application.

The following description and the annexed drawings set forth in detail certain illustrative aspects of the subject matter. These aspects are indicative, however, of but a few of the various ways in which the principles of the subject matter may be employed and the present subject matter is intended to include all such aspects and their equivalents. Other objects, advantages and novel features of the subject matter will become apparent from the following detailed description of the subject matter when considered in conjunction with the drawings.

### BRIEF DESCRIPTION OF THE SEVERAL VIEWS OF THE DRAWINGS

The foregoing and other features and advantages of the present subject matter will become apparent to those skilled in the art to which the present subject matter relates upon reading the following description with reference to the accompanying drawings. It is to be appreciated that two copies of the drawings are provided; one copy with notations therein for reference to the text and a second, clean copy that possibly provides better clarity.

FIG. 1 depicts a portion of a patient's spinal column;

FIGS. 2A and 2B illustrates one exemplary embodiment of a spinal disc preparation tool;

FIG. 3A depicts one exemplary embodiment of a modular cutting head;

FIGS. 3B and 3C depict exploded views of the modular cutting head of FIG. 3A;

FIGS. 4A and 4C depict perspective and side views of the modular cutting head of FIG. 3A in a closed position;

FIGS. 4B and 4D depict perspective and side views of the modular cutting head of FIG. 3A in an open position

FIG. 5 depicts the modular cutting head of FIG. 3A inserted into a disc space between adjacent vertebrae;

FIGS. 6A through 6D depict various exemplary embodiments of cutting surfaces;

FIGS. 7A through 7E depict various alternative cutting surface arrangements on a surgical cutting tool; and

FIGS. 8A and 8B depict alternative nesting cutting surface arrangements.

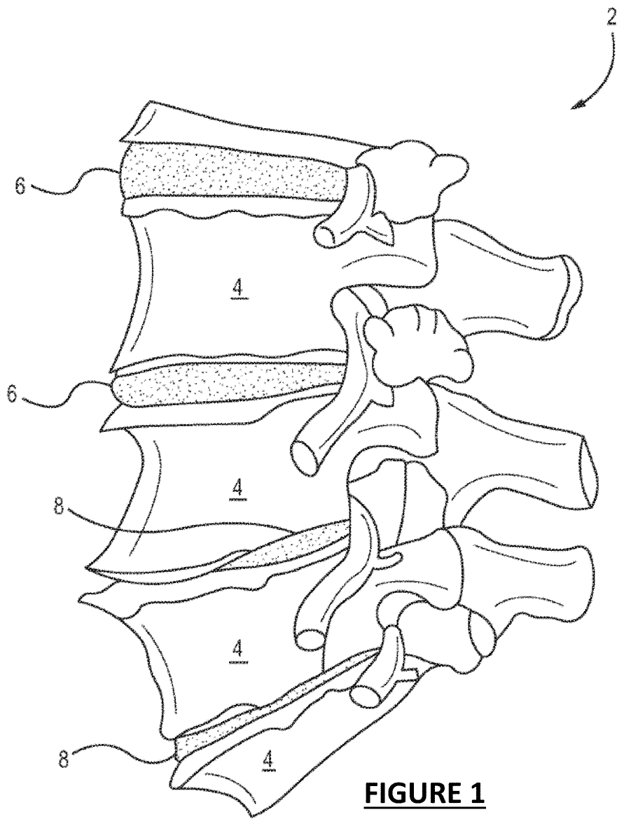

### DETAILED DESCRIPTION OF THE INVENTION

Certain exemplary embodiments will now be described to provide an overall understanding of the principles of the structure, function, manufacture, and use of the devices and methods disclosed herein. One or more examples of these embodiments are illustrated in the accompanying drawings. Those of ordinary skill in the art will understand that the devices and methods specifically described herein and illustrated in the accompanying drawings are non-limiting exemplary embodiments and that the scope of the present invention is defined solely by the claims. The features illustrated or described in connection with one exemplary embodiment may be combined with the features of other embodiments. Such modifications and variations are intended to be included within the scope of the present invention.

The present invention provides various methods and devices for preparing an intervertebral disc space. A person skilled in the art will appreciate that the methods and devices described herein can also be used for the removal of soft tissue, muscle, fascia, or ligaments prior to entering the disc space. In general, a spinal disc preparation tool is provided that is adapted to cut tissue using one or more of an upper and/or lower jaw at a distal end of the tool.

The present subject matter relates generally to devices useful during surgical procedures for the repair, fixation and/or support of vertebrae. In particular, the present subject matter relates to surgical tools preparing an anatomical location to receive an implant device for the spine, although various other anatomical locations of the body may benefit from various features of the present invention. The spinal column of vertebrates provides support to bear weight and protection to the delicate spinal cord and spinal nerves. The spinal column includes a series of vertebrae stacked on top of each other. There are typically seven cervical (neck), twelve thoracic (chest), and five lumbar (low back) segments. Each vertebra has a cylindrical shaped vertebral body in the anterior portion of the spine with an arch of bone to the posterior, which covers the neural structures. Between each vertebral body is an intervertebral disk, a cartilaginous cushion to help absorb impact and dampen compressive forces on the spine. To the posterior, the laminar arch covers the neural structures of the spinal cord and nerves for protection. At the junction of the arch and anterior vertebral body are articulations to allow movement of the spine.

Various types of problems can affect the structure and function of the spinal column. These can be based on degenerative conditions of the intervertebral disk or the articulating joints, traumatic disruption of the disk, bone or ligaments supporting the spine, tumor or infection. In addition, congenital or acquired deformities can cause abnormal angulation or slippage of the spine. Anterior slippage (spondylolisthesis) of one vertebral body on another can cause compression of the spinal cord or nerves. Patients who suffer from one of more of these conditions often experience extreme and debilitating pain, and can sustain permanent neurological damage if the conditions are not treated appropriately.

Alternatively or in addition, there are several types of spinal curvature disorders. Examples of such spinal curvature disorders include, but need not be limited to, lordosis, kyphosis and scoliosis.

One technique of treating spinal disorders, in particular the degenerative, traumatic and/or congenital issues, is via surgical arthrodesis of the spine. This can be accomplished by removing the intervertebral disk and replacing it with implant(s) and/or bone and immobilizing the spine to allow the eventual fusion or growth of the bone across the disk space to connect the adjoining vertebral bodies together. The stabilization of the vertebra to allow fusion is often assisted by the surgically implanted device(s) to hold the vertebral bodies in proper alignment and allow the bone to heal, much like placing a cast on a fractured bone. Such techniques have been effectively used to treat the above-described conditions and in most cases are effective at reducing the patient's pain and preventing neurological loss of function.

Because of various natural anatomical variation in the patient population, as well as spinal curvature disorders and/or contour issues present on the surfaces of many vertebrae, it may be difficult for a surgeon to properly prepare one or more surfaces of a vertebral body and/or disc space for placement of an implant. As such, there is need for further improvement. The present subject matter is such improvement.

The present subject matter will now be described with reference to the drawings, wherein like reference numerals are used to refer to like elements throughout. It is to be appreciated that the various drawings are not necessarily drawn to scale from one figure to another nor inside a given figure, and in particular that the size of the components may be arbitrarily drawn for facilitating the understanding of the drawings. In the following description, for purposes of explanation, numerous specific details are set forth in order to provide a thorough understanding of the present subject matter. It may be evident, however, that the present subject matter can be practiced without these specific details. Additionally, other embodiments of the subject matter are possible and the subject matter is capable of being practiced and carried out in ways other than as described. The terminology and phraseology used in describing the subject matter is employed for the purpose of promoting an understanding of the subject matter and should not be taken as limiting.

The various surgical devices described herein, as well as any portions or combination of portions thereof, can be constructed from a variety of surgically acceptable materials, including radiopaque and/or radiolucent materials, other materials or combinations of such materials. Radiolucent materials can include, but are not limited to, polymers, carbon composites, fiber-reinforced polymers, plastics, combinations thereof and the like. Radiopaque materials are traditionally used to construct devices for use in the medical device industry. Radiopaque materials can include, but are not limited to, metal, aluminum, stainless steel, titanium, titanium alloys, cobalt chrome alloys, combinations thereof and the like.

Various embodiments disclosed herein include cutting and/or scraping tools for preparing bony surfaces, such as the endplates of adjacent vertebral bodies, for placement of implants, graft materials and/or the like during surgery of the spinal column and/or other body locations. In many surgeries, disc end plate preparation can be essential for removing the undesirable disc elements and scoring the end plate for bony fusion to take place. Removal of disc elements—particularly those attached to the end plate—can be time consuming as both end plates typically are prepared while the vertebral bodies are being distracted and/or the preparation may be performed under narrow disc height, one at a time.

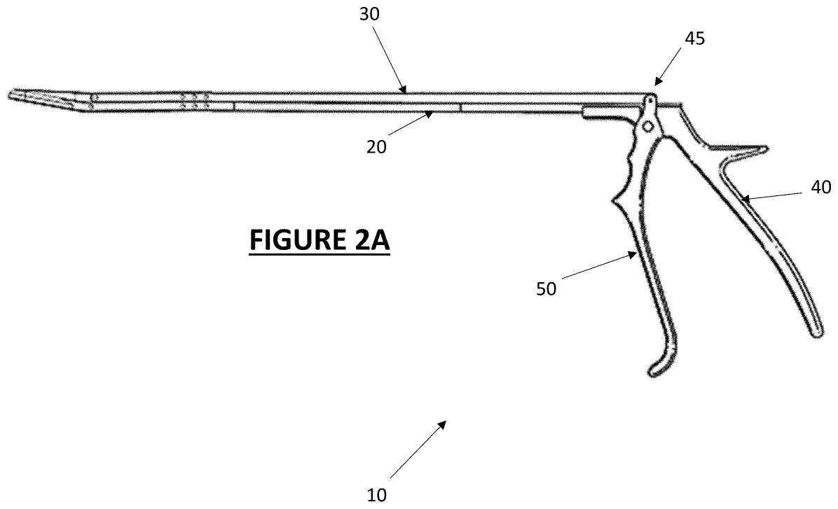

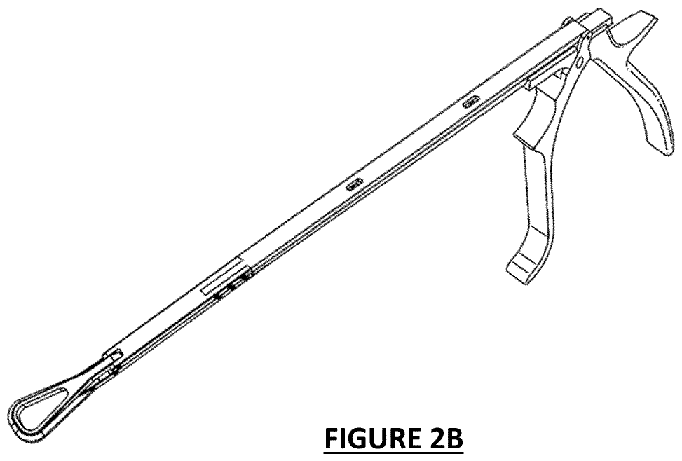

The present invention provides various methods and devices for preparing an intervertebral disc space. A person skilled in the art will appreciate that the methods and devices described herein can also be used for the removal of soft tissue, muscle, fascia, or ligaments prior to entering the disc space. In general, a spinal disc preparation tool is provided that is adapted to cut tissue between an upper and lower jaw at a distal end of the tool. FIGS. 2A and 2B illustrates one exemplary embodiment of a spinal disc preparation tool 10 that generally includes a pair of first and second elongate shafts 20 and 30, the first shaft 20 connected to a hand grip 40 and the second shaft 30 connected through a rotating connection 45 to an actuating lever 50, the actuating lever 50 being pivotally mounted to the hand grip 40 in a known manner. The first and second elongate shafts 20 and 30 are desirably slidably connected to one another (such as, for example, using a tab and groove connection or similar known arrangement) so as to allow the first elongate shaft 20 to move longitudinally relative to the second elongate shaft 30 in a known manner.

The elongate shafts can have a variety of configurations, and can be rigid or flexible depending on the intended use. In an exemplary embodiment, the elongate shafts can be adapted to be positioned within a body cavity and thus can have a length sufficient to allow the distal end of the shafts to be positioned within the body while the proximal end remains external to the body to allow a user to grasp the handle and operate an actuator extending from the proximal end of the tool. One skilled in the art will appreciate that the elongate shafts and/or other components of the tool can be made from a variety of biocompatible materials that have properties sufficient to enable the various components to be inserted and moved within the body.

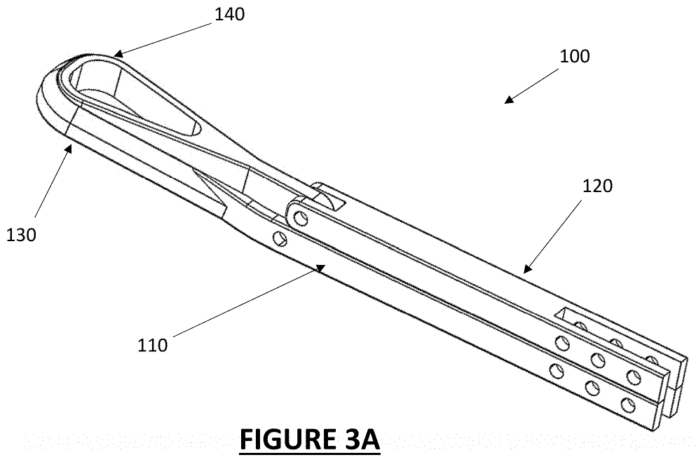

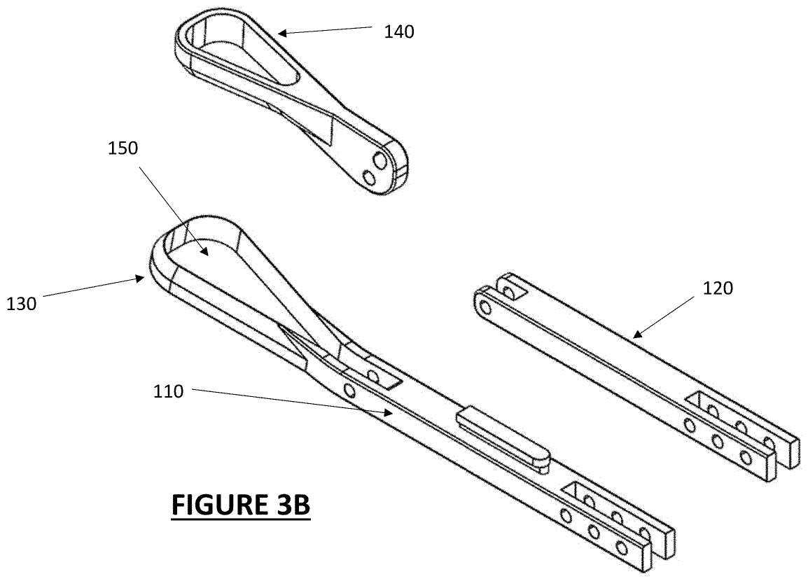

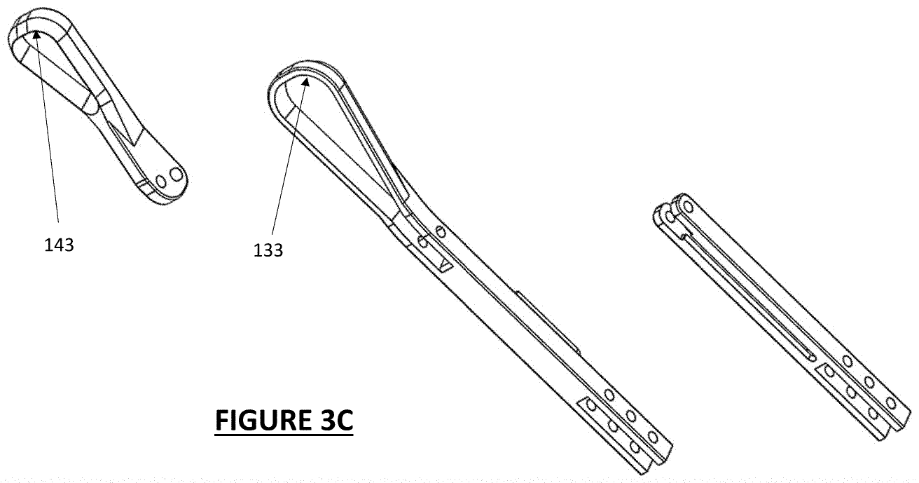

Disposed at the distal ends of the first and second elongate shafts 20 and 30 is a modular cutting head. As best seen in FIG. 3A, a first embodiment of a cutting head 100 can include a first base member 110 which can be connected at a proximal end to the first elongate shaft 20 and a second actuating shaft 120 which can be connected at a proximal end to the second elongate shaft 40. A first cutting head 130 having a distal central opening 150 is disposed on a distal portion of the first base member 110, and a second cutting head 140 is rotatably connected to a distal end of the second actuating shaft. In various embodiments, the various components can be connected using a variety of connection methods, including removable pins, screw, rivets and/or other connections known in the art. For example, the various components described herein could be connected using Morse taper pins (not shown), compression shafts and/or or similar devices.

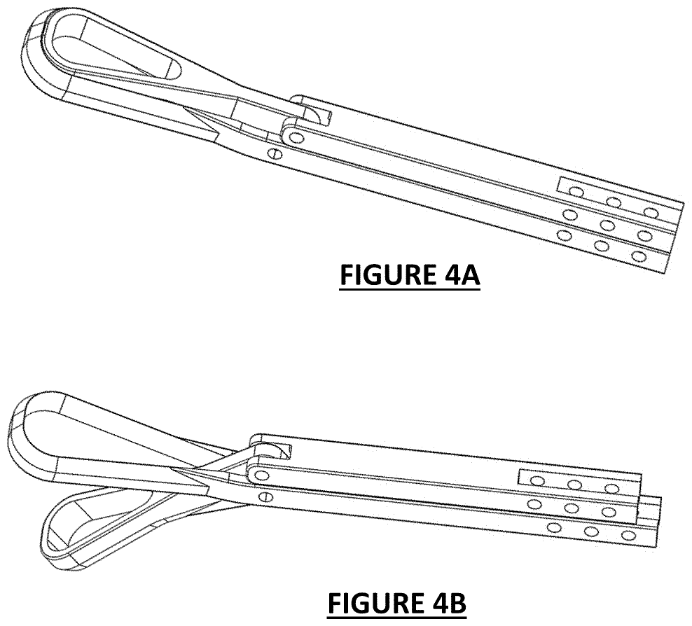

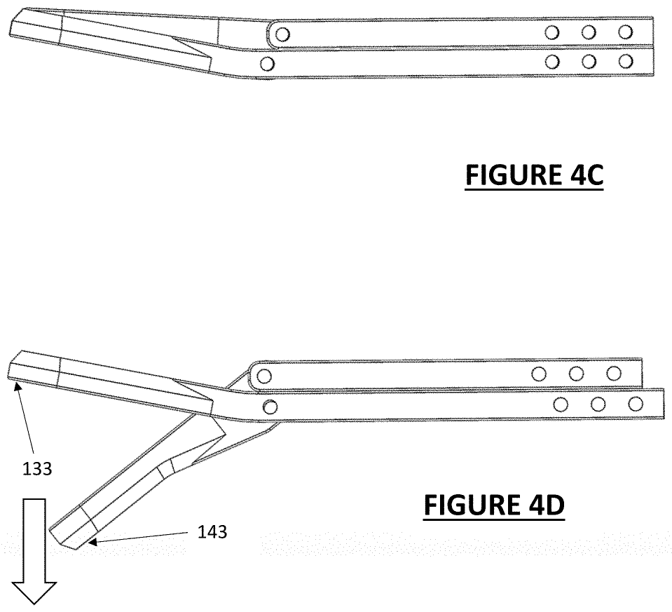

FIGS. 4A through 4D depict various views of the cutting head of FIG. 3A at rest or in a closed position (FIGS. 4A and 4C) and when opened or actuated (FIGS. 4B and 4D). In use, when the actuating lever 50 can be drawn towards the hand grip 40 by a user, this drawing motion causing the first elongate shaft 20 to slide distally relative to the second elongate shaft 30, in turn causing the second actuating shaft 120 to move distally relative to the first base member 110. Because the second cutting head 140 is rotatably connected to both the first base member 110 and the second actuating shaft 120, the relative motion therebetween causes the second cutting head 140 to rotate in a counterclockwise manner, forcing the distal tip of the second cutting head through and/or out of a central opening and downward relative to the first cutting head 130 (see FIGS. 4B and 4D). Desirably, the user can increase and/or decrease the amount that the actuating lever 50 is drawn towards the hand grip 40 (or the hand pressure used therefor), which will desirably increase and/or decrease the amount that the distal tip of the second cutting head 140 extends through the central opening and downward relative to the first cutting head 130. Accordingly, the various embodiments disclosed herein desirably provide an intuitive design which grants a surgeon significant amounts of tactile feedback, which allows the surgeon to loosen or tighten their grip and/or handle squeeze as the tip(s) scrapes the uneven concave endplates that varies from patient to patient.

In various embodiments, either or both of the first and second cutting heads can include at least one cutting surface, with the at least one cutting surface typically shielded by other surfaces or structures of the first and second cutting heads when the device is in a closed or “at rest” position (i.e., FIGS. 4A and 4C), but when the device is opened or actuated, the at least one cutting surface is then exposed to allow the device to cut surrounding tissues (i.e., FIGS. 4A and 4C). For example, the second cutting head 140 can include a lower cutting surface 143 which is typically shielded by a lower surface 133 of the first cutting head 130, with the lower cutting surface 143 of the second cutting 140 becoming exposed when the tool is actuated.

If desired, both of the first and second cutting heads can include cutting surfaces that are shielded when the tool is closed, but which are exposed when the tool is actuated. For example, the first cutting head 130 may include an upwardly facing cutting surface, and the second cutting head 140 can include downward facing cutting surface, if desired. When the handle of the tool is squeezed, the cutting tips can open up and splay outward. Desirably, the outer edges of each cutting tip (which may be formed in the shape of a ring curette, if desired) are sharp, thus taking material out of both the top and bottom vertebral endplates to prepare the bony surfaces in a highly effective manner while requiring fewer surgical steps (and desirably reducing surgical time).

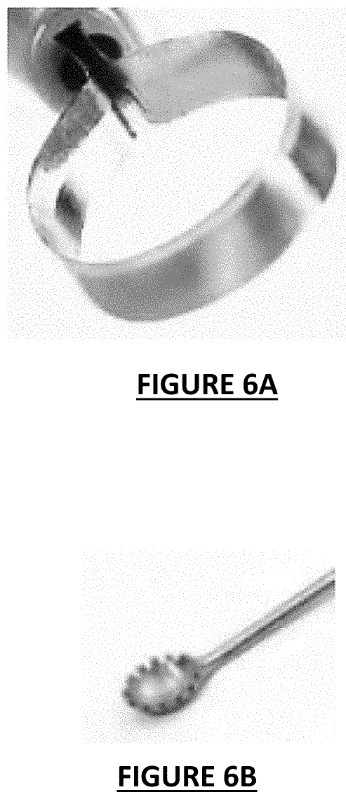

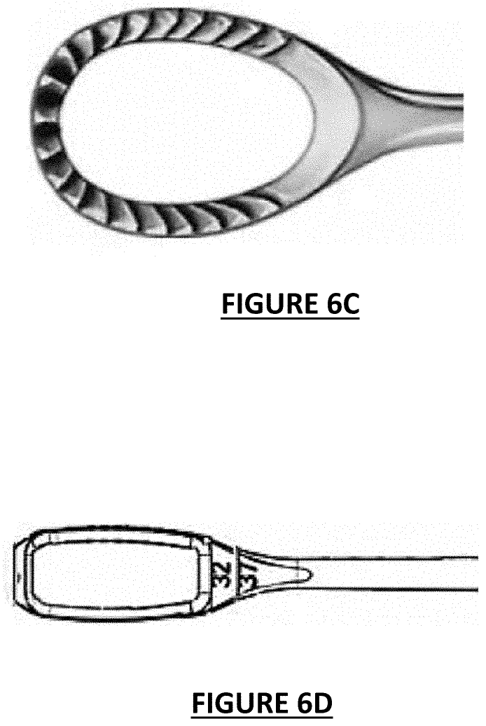

In various alternative embodiments, the cutting tips could be biased such that the cutting edges are slightly and/or somewhat recessed when the tool is in a closed or “at-rest” position (i.e., when the cutting tips are not splayed). For example, FIG. 6A depicts a prep tool having cutting surfaces on inner edges of the distal rings, wherein the rings pass through each other when the handle is squeezed or otherwise actuated, exposing the cutting surfaces to the surrounding tissues. FIGS. 6B and 6C provide exemplary designs for the cutting ring surfaces, although a wide variety of shapes known in the art for such cutting tools could be utilized, including symmetric and/or nonsymmetric shapes for either or both of the upper and/or lower rings (including various combinations thereof).

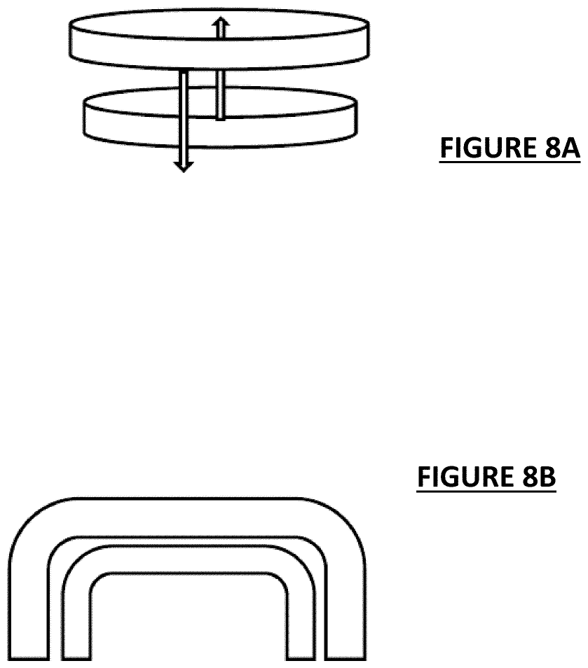

In various disclosed embodiments, one of the cutting surfaces of the tool could desirably “nest” or otherwise stack or engage with the adjacent cutting head, with one of the cutting heads fitting at least partially or fully inside of the other. Such arrangements could include fully nesting cutting surfaces such as shown in FIGS. 8A and 8B, as well as partially nesting designs. If desired, one cutting surface could comprise a “ring” surface, while the opposing cutting surface could be a solid plate, cylinder or cup shape which passes into and/or through the ringed surface.

In various alternative embodiments, the elongate shafts of the tool could optionally include features to allow the cutting head to reach a contralateral side of the vertebral disc space. In one embodiment, the cutting head and/or a distal portion of the elongated shafts could be pre-bent to form an arc in a variety of directions, but in at least one embodiment the arc could be such that the cutting head resides in substantially the same plane as the handle of the tool. In another embodiment, the elongate shafts could include features to allow a distal portion of the elongate shafts to articulate to redirect the distal portion of the elongate shaft. For example, the elongate shafts and/or the cutting head could include an articulation joint formed at a location proximal to the cutting surfaces, or at some point between the handle and the cutting surfaces. The articulation joint could allow portions of the elongate shafts to pivot side-to-side along a single plane, or in other embodiments along multiple planes, relative to the handle and/or the cutting head. Articulation could be controlled by a trigger, knob, or other actuation mechanism on the handle, or actuation can be passively controlled by applying a force to the distal portion, e.g., with tissue. A person skilled in the art will appreciate that various articulation mechanisms known in the art can be used to allow articulation of the distal portion of the elongate shaft. While not shown, the elongate shaft can also optionally be configured to rotate relative to the handle, and various rotation mechanisms known in the art can be used.

In various embodiments, the first and second cutting surfaces associated with the distal end of the tool can have a variety of configurations. In the illustrated embodiment, the cutting surfaces can be ring-shaped, or the surfaces could have a generally oval or elongated square or triangular shape that extends distally from the distal end of the tool. The first or second cutting surface could be formed from an opening or concavity formed in one of the elongate shafts, if desired, that may be adapted to receive cut tissue, or the first or second cutting surface could be a separate component that is fixedly or removably attached to the distal end of the tool or cutting head. A person skilled in the art will appreciate that the various cutting surfaces and/or related components described herein can be attached to the elongate shafts in any manner, including by removable and replaceable fitting to a variety of cutting heads and/or cutting surfaces to be selectively attached to the tool. In such a case, the cutting head and/or components thereof could include modular connections at a variety of locations to allow replacement for wear and tear and/or breakage.

If desired, the cutting head can include upper and/or lower jaws that include tissue cutting surface features that can be configured to prepare a vertebral endplate for insertion of an implant between adjacent vertebrae after the removal of the cut tissue. For example, the tissue cutting surface features can be configured to remove tissue along a surface of a vertebral endplate, or they can be adapted to cut into the surface of the vertebral endplate. The tissue cutting surface features can be formed on either or both of the upper and lower jaws and on any location of the jaws in order to facilitate preparation of the endplate. The tissue cutting surface features can also have a variety of configurations. In some exemplary embodiments shown in FIGS. 6B and 6C, the tissue cutting surface features are in the form of a rasp. The rasp can include a plurality of teeth adapted to remove tissue and cut into the surface of the vertebral endplate. The rasp can include any number of teeth, and the teeth can be formed on any location of upper and/or lower jaw. In another exemplary embodiment shown in FIGS. 6A and 6D, the tissue cutting surface features can be in the form of a curette formed on an exterior surface of a jaw. The curette can be in the form of a cylindrical or angular protrusion formed on upper or lower jaw that is adapted to cut and/or scrape tissue from vertebral endplates. A person skilled in the art will appreciate that any type of tissue cutting surface features can be formed on the upper and/or lower jaws at any location thereon for preparing vertebral endplates.

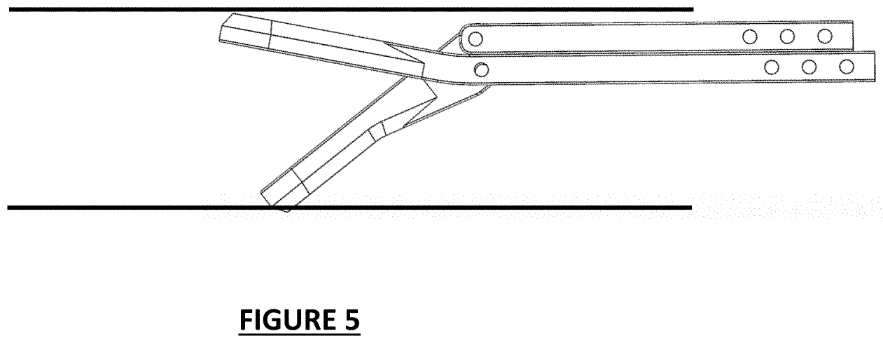

The present invention also provides methods for cutting tissue. In one exemplary embodiment shown in FIG. 5, the cutting head at the distal end of the tool can be inserted into the disc space between adjacent vertebrae for cutting the tissue therebetween, for example, a spinal disc which can be removed to be replaced with a spinal disc implant. The distal end of the elongate shaft can be positioned adjacent to the tissue to be cut such that the upper and lower jaws are placed adjacent the tissue. A distal portion of the elongate shafts might also be articulated (not shown) to help facilitate positioning of the upper and lower jaws relative to the tissue. The actuator pivotally coupled to the handle of the tool can be actuated to cause one jaw element to move and pivot relative to the other to cause the distal ends of the tool to spread apart and cause the one or more cutting surfaces to contact and/or abrade adjacent tissues.

In some instances, the use of the tool in a narrow disc space may allow the surgeon to use the opposing vertebral walls to increase pressure of the cutting surfaces against one or both walls of the vertebrae, such that the top and bottom end plates may be simultaneously engaged by cutting surfaces during disc removal.

In one exemplary embodiment, the tool can include a cutting surface which faces towards and engages with one of the upper and/or lower vertebral bodies, while the opposing surface of the tool (in contact with the opposing vertebral body) may have a smooth or non-cutting surface facing towards the vertebral body. In such a case, the non-cutting surface may be used as a leverage point against the non-cut vertebral surface to increase the depth and/or quantity of cut that is being made by the cutting surface. Once the desired surface if prepared, the tool could be inverted and the remaining vertebral surface prepared in a similar manner.

If desired, as a tissue is being cut and/or removed, for example from the disc space between adjacent vertebrae, one or more opposing tissue cutting surface features formed on the upper and/or lower jaws could be utilized to cut the vertebral endplates or scrape tissue therefrom to prepare the endplates for insertion of a spinal disc implant into the disc space between the adjacent vertebrae.

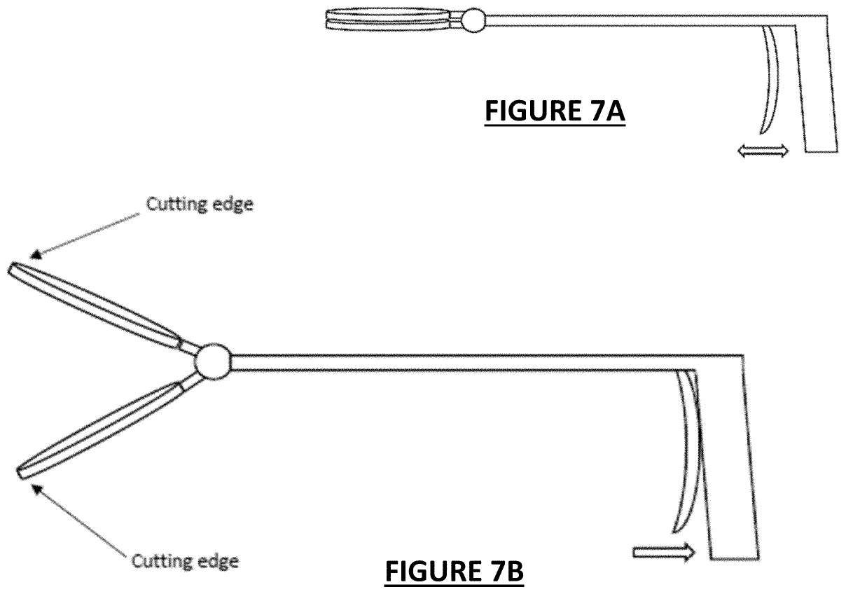

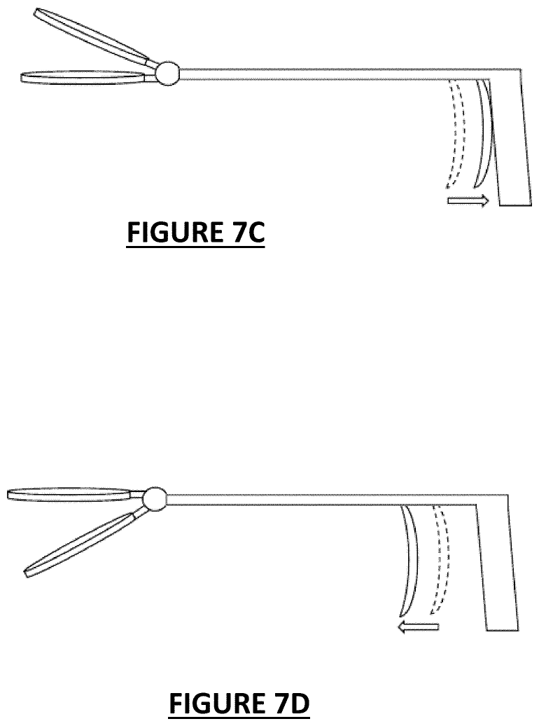

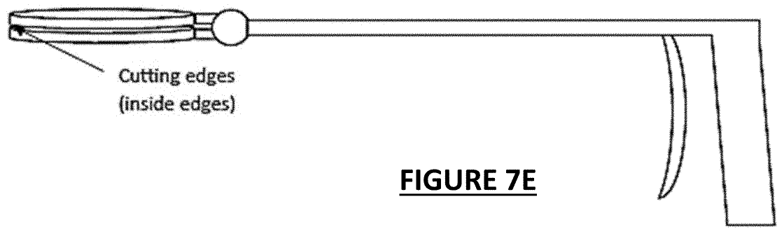

FIGS. 7A and 7B depict another exemplary embodiment of a surgical tool design that can accommodate asymmetric extension and/or contraction of the cutting surfaces, which desirably allows a surgeon to prepare one or both of the vertebral surfaces as desired without requiring removal of the cutting tool.

If desired, the cutting surfaces disclosed herein could include “down-biting” or “up-biting” surfaces, or combinations thereof, including the incorporation of multiple cutting surfaces on a single cutting or arm element.

In various embodiments, the cutting surfaces could include an arrangement where only a single cutting surface moves relative to the other cutting surface (i.e., top motion and bottom fixed, or bottom motion and top fixed designs), or both cutting surfaces could be arranged and/or configured to move relative to other tool components.

If desired, the cutting surface of a given tool may be continuous and/or discontinuous in the shape and/or disposition of the sharp cutting edge of the surface, including the use of planar or curved cutting surfaces and/or toothed surfaces. The geometry of the cutting surface(s) and/or cutting jaws may also vary, including circular, oval, square, rectangular, triangular, polygonal, multi-polygonal, concave, convex and/or other shapes known in the art.

In various embodiments, a surgical tool kit could include one or more replacement components for the system, including individual replacement cutting surface components or a modular replacement cutting head, if desired. The various components of these systems could optionally be provided in kit form, with a medical practitioner having the option to select an appropriately sized and/or shaped cutting head to attach to a surgical tool to address a desired surgical situation.

Note that, in various alternative embodiments, variations in the position and/or relationships between the various figures and/or modular components are contemplated, such that different relative positions of the various modules and/or component parts, depending upon specific module design and/or interchangeability, may be possible. In other words, different relative adjustment positions of the various components may be accomplished via adjustment in separation and/or surface angulation of one of more of the components to achieve a variety of resulting tool configurations and/or sizes, thereby accommodating virtually any expected anatomical variation. For example, variation of the thicknesses and/or separation distance between the cutting surfaces (i.e., optionally without altering the angulation of the cutting surfaces) can desirably cause an increase or decrease in the size or “height” of the space created for the implant, due to changes in the z-axis positioning of the components which engage the adjacent vertebrae. Concurrently, alterations in the “tilt angle” or angulation of one or both of the cutting surfaces or other components in the medial-lateral (i.e., rotation about a y-axis) and/or anterior-posterior (i.e., rotation about an x-axis) axes of the tool will allow the tool to be utilized accommodate a wide variety of natural and/or surgically altered surfaces of the spine. Moreover, various complex combinations (at various amounts) of comparative lateral (e.g., left-right) tilt and fore-aft (e.g., anterior-posterior) tilt can be accomplished, with or without concurrent adjustments in the various cutting surfaces.

The various embodiments of a surgical tool disclosed herein can be configured to interact with two bone vertebrae of a spine or other anatomical locations. The spine may have any of several types of spinal curvature disorders which are sought to be treated. Examples of such spinal curvature disorders include, but need not be limited to, lordosis, kyphosis, scoliosis and/or low and/or high velocity fractures, among other pathologies.

In various exemplary scenarios, the disclosed surgical tools can be used in conjunction with various implant devices utilized to fix and/or secure adjacent vertebrae that have had cartilaginous disc between the vertebrae replaced with fusion material that promotes the fusion of the vertebrae, such as a graft of bone tissue. Also, such can be accomplished even when dealing with a spinal curvature disorder (e.g., lordosis, kyphosis and scoliosis).

Of course, method(s) for manufacturing the surgical device and implanting an implant device into a spine are contemplated and are part of the scope of the present application.

While embodiments and applications of the present subject matter have been shown and described, it would be apparent to those skilled in the art that many more modifications are possible without departing from the inventive concepts herein. The subject matter, therefore, is not to be restricted except in the spirit of the appended claims.

All references, including publications, patent applications, and patents, cited herein are hereby incorporated by reference to the same extent as if each reference were individually and specifically indicated to be incorporated by reference and were set forth in its entirety herein.

The various headings and titles used herein are for the convenience of the reader, and should not be construed to limit or constrain any of the features or disclosures thereunder to a specific embodiment or embodiments. It should be understood that various exemplary embodiments could incorporate numerous combinations of the various advantages and/or features described, all manner of combinations of which are contemplated and expressly incorporated hereunder.

The use of the terms “a” and “an” and “the” and similar referents in the context of describing the invention are to be construed to cover both the singular and the plural, unless otherwise indicated herein or clearly contradicted by context. The terms “comprising,” “having,” “including,” and “containing” are to be construed as open-ended terms (i.e., meaning “including, but not limited to,”) unless otherwise noted. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., i.e., “such as”) provided herein, is intended merely to better illuminate the invention and does not pose a limitation on the scope of the invention unless otherwise claimed. No language in the specification should be construed as indicating any non-claimed element as essential to the practice of the invention.

Preferred embodiments of this invention are described herein, including the best mode known to the inventor for carrying out the invention. Variations of those preferred embodiments may become apparent to those of ordinary skill in the art upon reading the foregoing description. The inventor expects skilled artisans to employ such variations as appropriate, and the inventor intends for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

1. A spinal disc preparation tool, comprising:
a stationary arm, the stationary arm having a proximal end and an opposing distal end; a movable arm slidably coupled to the stationary arm, the movable arm having a proximal end and an opposing distal end;
a handle operatively coupled to the stationary arm and the movable arm; and a tip assembly removably coupled in an assembled configuration to the distal end of the stationary arm and the distal end of the movable arm, the tip assembly comprising:
a first tip; and
a second tip rotatably coupled to the first tip, the second tip slidably movable with respect to the first tip between an open position and a nested position;
wherein the second tip includes a cutting surface which is shielded by the first tip when the second tip is in the nested position, but the cutting surface is not shielded by the first tip when the second tip is in the open position.

2. The spinal disc preparation tool of claim 1, further comprising a locking mechanism configured to removably couple the first tip to the stationary arm and removably couple the second tip to the movable arm.

3. The spinal disc preparation tool of claim 2, wherein the locking mechanism comprises:
a first attachment member disposed on a proximal end of the first tip; and
a first receiving section disposed on the stationary arm, the first receiving section configured to receive the first attachment member.

4. The spinal disc preparation tool of claim 3, wherein the locking mechanism comprises:
a second attachment member disposed on the distal end of the second arm; and a second receiving section disposed on the second tip, the second receiving section configured to receive the second attachment member.

5. The spinal disc preparation tool of claim 1, wherein the first tip includes a non-cutting surface.

6. The spinal disc preparation tool of claim 1, wherein the first tip includes a cutting surface.

7. The spinal disc preparation tool of claim 1, wherein the first tip includes a lumen extending therethrough, and the second tip nests fully within the lumen.

8. The spinal disc preparation tool of claim 1, wherein the first tip includes a lumen extending therethrough, and the second tip nests partially within the lumen.

9. The spinal disc preparation tool of claim 1, wherein the first tip comprises a circular ring.

10. The spinal disc preparation tool of claim 1, wherein the second tip comprises a circular ring.

11. The spinal disc preparation tool of claim 1, wherein the second tip comprises a toothed surface.

12. The spinal disc preparation tool of claim 1, wherein the second tip comprises a rasp surface.
